(19) United States
(12) Patent Application Publication
(10) Pub. No.: US 2008/0294176 A1
(43) Pub. Date: Nov. 27, 2008

(54) HAIR REMOVAL APPARATUS

(75) Inventors: Tetsuro HASHIGUCHI, Hikone (JP); Kenichi MURAKI, Hikone (JP)

Correspondence Address: GREENBLUM & BERNSTEIN, P.L.C., 1950 ROLAND CLARKE PLACE, RESTON, VA, 20191, US

(73) Assignee: MATSUSHITA ELECTRIC WORKS, LTD., Osaka (JP)

(21) Appl. No.: 12/029,690

(22) Filed: Feb. 12, 2008

(30) Foreign Application Priority Data
Feb. 23, 2007 (JP) 2007-043360

(51) Int. Cl. A61B 17/50; B26B 19/00; B26B 19/14

(52) U.S. Cl. 606/133; 606/211; 30/34.05; 30/43.5

(57) ABSTRACT

A hair removal apparatus includes three plucking rollers configured to rotate on respective roller shafts which extend generally parallel to a skin surface during epilation, each plucking roller having tweezing mechanisms for grasping hairs, and a driver for turning each plucking roller on a rotary axis which is kept generally perpendicular to the skin surface. The tweezing mechanisms pull the grasped hairs out of the skin as the plucking rollers are slid therealong. The three plucking rollers can protrude and retract in a direction generally perpendicular to the skin surface independently of one another and swing in directions along the skin surface independently of one another.

### BACKGROUND OF THE INVENTION

1. Field of the Invention

The present invention relates to a hair removal apparatus for removing unwanted body hair by pulling the hair out of the skin.

2. Description of the Related Art

Japanese Unexamined Patent Publication No. 1998-151020 describes a conventionally known example of a hair removal apparatus provided with an extractor for pulling hairs out of human skin by sliding the apparatus along the skin surface so that the extractor grips and removes the hairs. The hair removal apparatus proposed in the Publication includes a plurality of tweezing discs (extractor) to provide a capability to treat an increased epilation area.

It has conventionally been desired for the aforementioned type of hair removal apparatuses to ensure an increased contact area between individual extractors and the skin surface yet preventing a reduction in epilation efficiency.

### SUMMARY OF THE INVENTION

The present invention is intended to achieve the aforementioned object. Accordingly, it is a specific object of the invention to provide a hair removal apparatus having a plurality of extractors which can treat an increased epilation area yet preventing a reduction in epilation efficiency.

According to an aspect of the invention, a hair removal apparatus comprises a plurality of extractors each of which rotates on a first rotary axis extending in a direction generally parallel to a skin surface during epilation, each of the extractors having a tweezing mechanism for grasping hairs, and a driver for turning each of the extractors on a second rotary axis which is kept generally perpendicular to the skin surface. The plurality of extractors are configured to protrude and retract in a direction generally perpendicular to the skin surface independently of one another, and the hair removal apparatus pulls the grasped hairs out of the skin surface when moved with the extractors held in contact with the skin surface.

These and other objects, features and advantages of the invention will become more apparent upon a reading of the following detailed description in conjunction with the accompanying drawings.

### BRIEF DESCRIPTION OF THE DRAWINGS

FIG. 1 is a perspective view of a hair removal apparatus according to a preferred embodiment of the invention;

FIG. 2 is a side view of the hair removal apparatus shown in FIG. 1;

FIG. 3 is an exploded perspective view of the hair removal apparatus shown in FIG. 1;

FIG. 4 is a cross-sectional view of the hair removal apparatus taken along lines IV-IV shown in FIG. 1;

FIG. 5 is a cross-sectional view of the hair removal apparatus taken along lines V-V shown in FIG. 4;

FIG. 6 is a perspective view of a plucking roller of one of epilation units shown in FIG. 4;

FIG. 7 is a perspective view showing how motive power is transmitted by a first motive power transmission mechanism of the hair removal apparatus;

FIG. 8 is a perspective view showing how the motive power is transmitted by a second motive power transmission mechanism of the hair removal apparatus;

FIG. 9 is a side view showing the epilation units under normal conditions;

FIG. 10 is a side view showing the epilation units under floated conditions; and

FIG. 11 is a cross-sectional view corresponding to FIG. 4 showing one of the epilation units under the floated conditions.

### DETAILED DESCRIPTION OF THE PREFERRED EMBODIMENT OF THE INVENTION

A preferred embodiment of the invention is now described in detail with reference to the accompanying drawings.

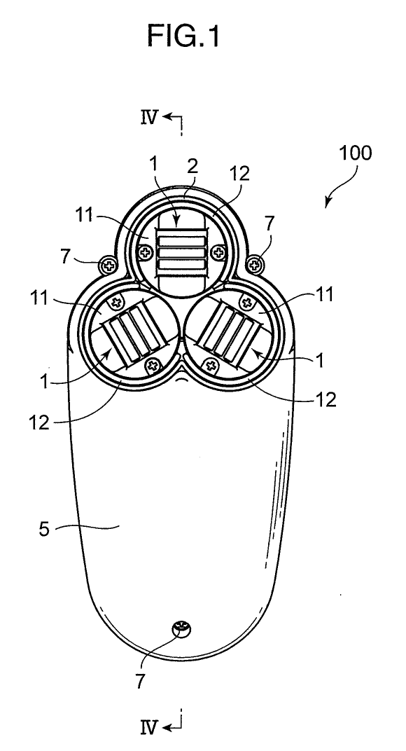

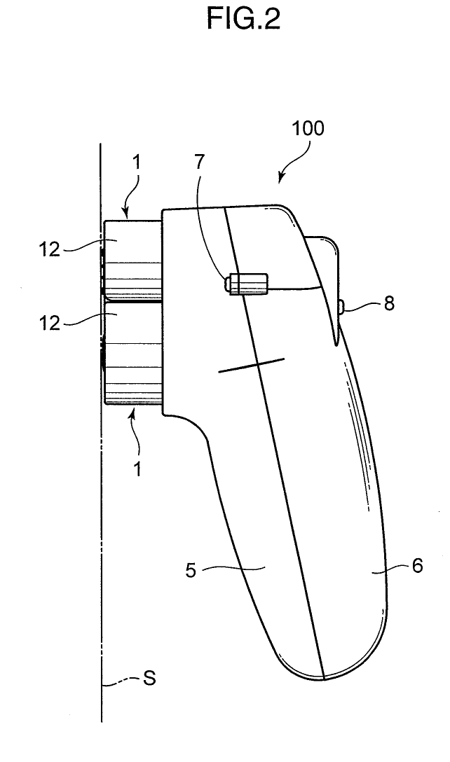

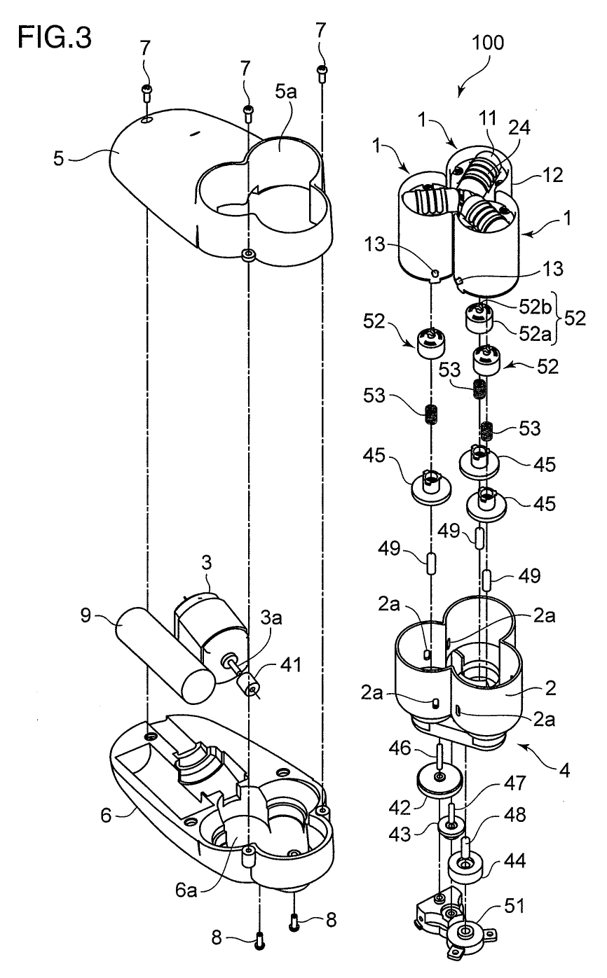

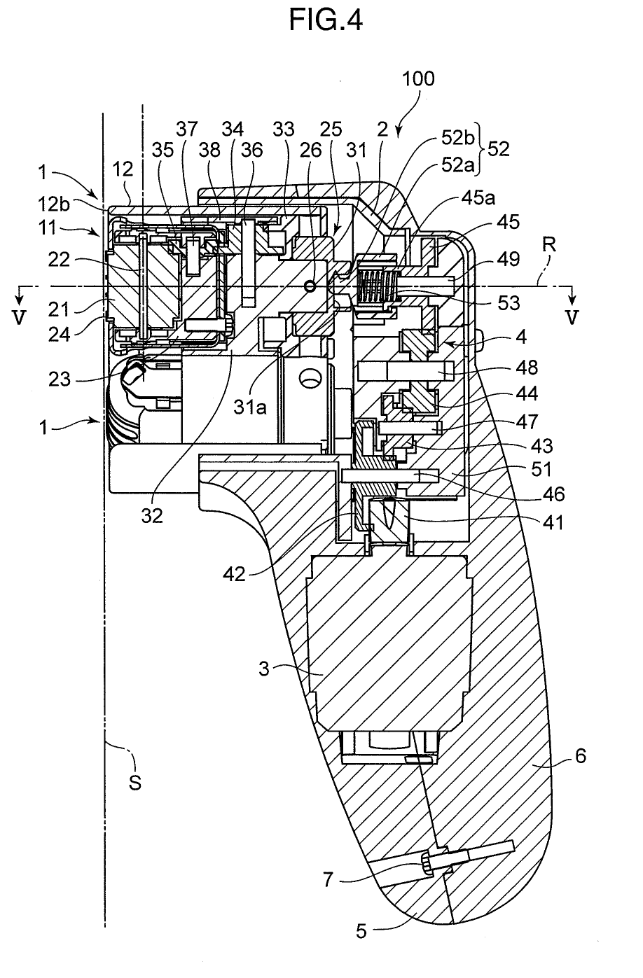

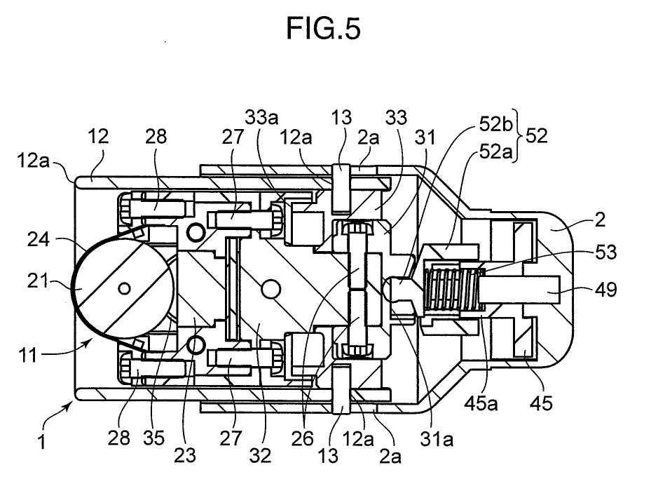

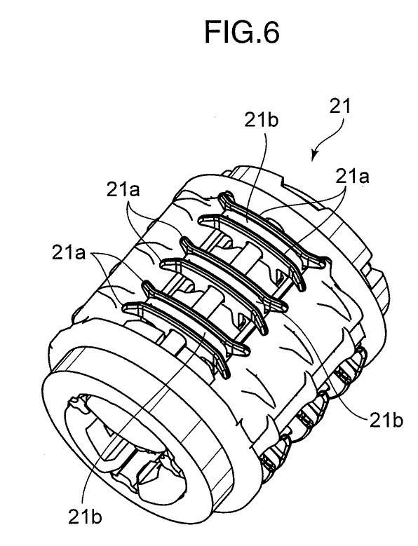

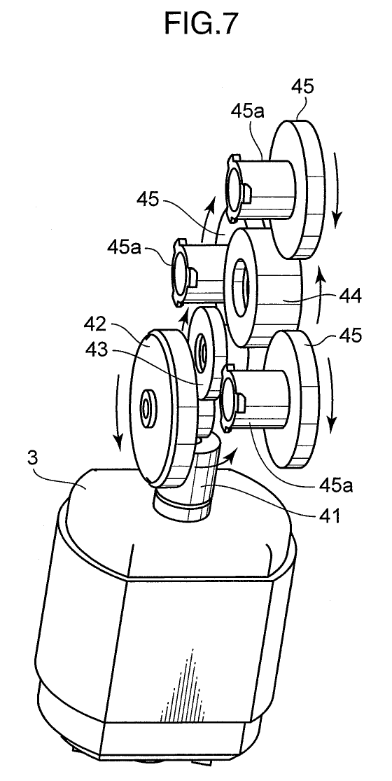

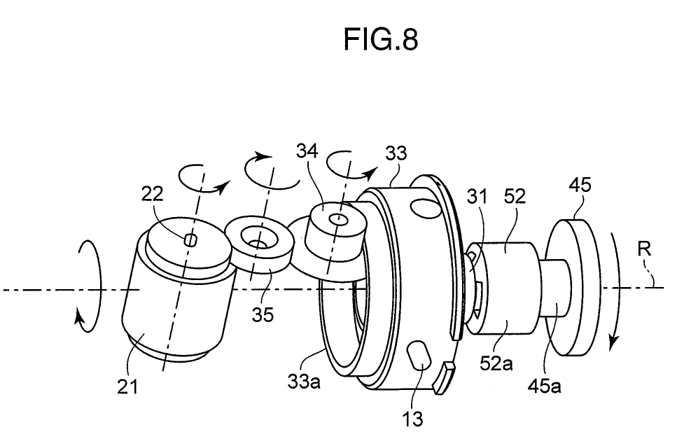

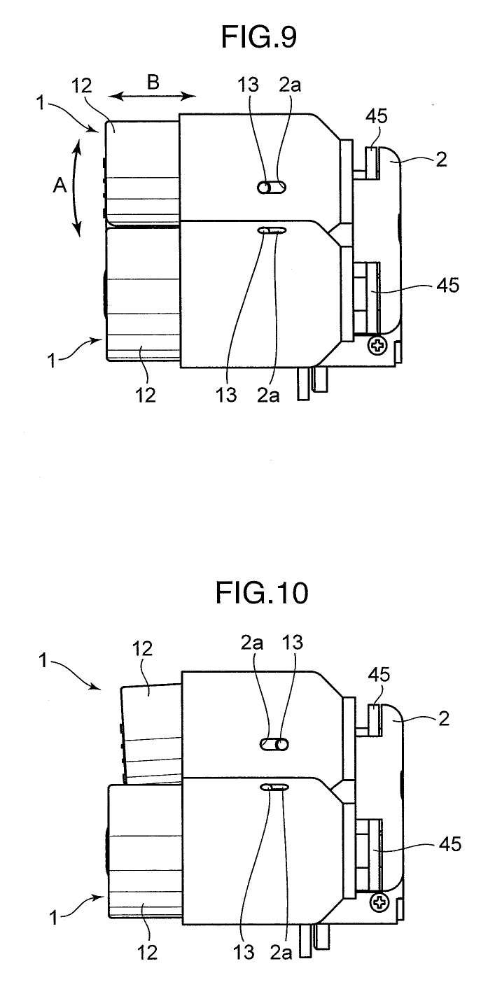

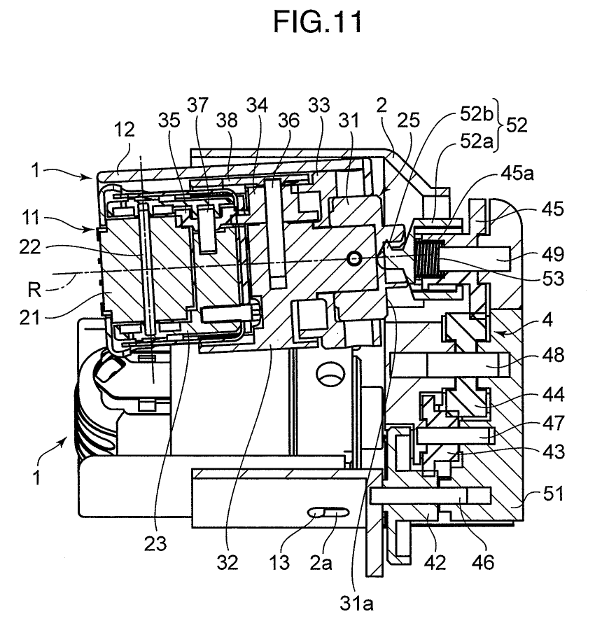

FIGS. 1 and 2 are a perspective view and a side view of a hair removal apparatus 100 according to a preferred embodiment of the invention, respectively, and FIG. 3 is an exploded perspective view of the hair removal apparatus 100 shown in FIG. 1. FIGS. 4 and 5 are cross-sectional views of the hair removal apparatus 100, and FIG. 6 is a perspective view of a plucking roller 21 of one of epilation units 1. FIGS. 7 and 8 are perspective views showing how motive power is generated and transmitted for driving the hair removal apparatus 100 and FIGS. 9 and 10 are side views showing the epilation units 1 under normal conditions and floated conditions, respectively. FIG. 11 is a cross-sectional view corresponding to FIG. 4 showing the epilation unit 1 under the floated conditions.

The hair removal apparatus 100 is configured to pull out unwanted body hairs, in particular those from arms, legs and underarms. Referring to FIGS. 1 to 3, the hair removal apparatus 100 includes three epilation units 1, a supportive housing 2 or casing for supporting the three epilation units 1, a motor 3 serving as a power source, a first motive power transmission mechanism 4 for transmitting a rotational driving force generated by the motor 3 to the epilation units 1, and a pair of outer covers 5, 6 incorporating all internal elements of the hair removal apparatus 100 together forming a structure grippable by a single hand. The two outer covers 5, 6 are fixed to each other by screws 7. There is formed an opening 5a in the outer cover 5 so that the three epilation units 1 are exposed outward toward a skin surface S as shown in FIG. 2. The motor 3 is connected to a battery 9 serving as a power supply.

The three epilation units 1 are closely located in such a way that central points of the epilation units 1 are positioned at vertices of a generally equilateral triangle in plan view as depicted in FIG. 1. Also, the three epilation units 1 are disposed to project toward the skin surface S generally by the same amount as depicted in FIG. 2. The hair removal apparatus 100 of this embodiment is configured such that the epilation units 1 can float (i.e., protrude and retract) in a direction generally perpendicular to the skin surface S and/or swing in a direction generally along the skin surface S independently of one another so that the individual epilation units 1 can be held well in close contact with the skin surface S having such bodily features as swells and hollows. As shown in FIGS. 3 to 5, each of the epilation units 1 includes a rotor 11, a sleeve 12 and a pair of pins 13 serving as a pivotal axis.

The rotor 11 is configured to rotate on an axis R shown in FIG. 4 due to the rotational driving force transmitted through the first motive power transmission mechanism 4 with the axis R kept generally perpendicular to the skin surface S. Specifically, the axis R of the rotor 11 passes approximately a central point of the plucking roller 21. The rotor 11 of each epilation unit 1 includes the aforementioned plucking roller 21, a roller shaft 22, a roller base 23, a protective plate 24 and a second motive power transmission mechanism 25.

Each of the plucking rollers 21 is supported rotatably on its own axis by the roller shaft 22. The plucking roller 21 of each rotor 11 carries a plurality of tweezing mechanisms 21a fitted on a curved outer surface, each tweezing mechanism 21a including a pair of platelike members arranged to extend side by side along a rotating direction of the plucking roller 21 with a narrow gap 21b formed between the platelike members to guide body hairs thereinto as shown in FIG. 6. These tweezing mechanisms 21a are configured to close the gaps 21b when brought into contact with the skin surface S, whereby the tweezing mechanisms 21a grasp the body hairs introduced into the gaps 21b.

The roller shaft 22 of each rotor 11 extends in a direction generally parallel to the skin surface S and each plucking roller 21 supported by the roller shaft 22 rotates on its own axis during epilation. When a user moves the hair removal apparatus 100 along the skin surface S, the rotating plucking rollers 21 grasp and pull out the body hairs.

As shown in FIG. 5, the roller base 23 supporting the roller shaft 22 of each rotor 11 is affixed to a later-described roller case 32 by screws 27.

The protective plates 24 of the rotors 11 lie on areas of the skin surface S with which the individual plucking rollers 21 go into contact. The protective plates 24 serve to protect the skin surface S by preventing the tweezing mechanisms 21a from contacting the skin surface S with excessive pressure. Each protective plate 24 is affixed to the roller base 23 by screws 28.

The second motive power transmission mechanism 25 of each rotor 11 is connected to the first motive power transmission mechanism 4 to transmit the rotational driving force supplied therefrom to the plucking roller 21 for rotating the same. As shown in FIG. 4, the second motive power transmission mechanism 25 includes a power input member 31, the aforementioned roller case 32, a crown gear 33 and epilation unit-side gears 34, 35.

The power input member 31 of each second motive power transmission mechanism 25 is a mechanical element of the epilation unit 1 to which the rotational driving force supplied from the first motive power transmission mechanism 4 is first transmitted. Each power input member 31 has a mating part 31a recessed in a U-shaped cross section which can flexibly engage a projecting part 52b of each of later-described cylindrical members 52 of the first motive power transmission mechanism 4. The power input member 31 thus configured turns on the aforementioned axis R due to the rotational driving force input from the first motive power transmission mechanism 4.

As the roller case 32 of each second motive power transmission mechanism 25 is joined to the power input member 31 by screws 26, the roller case 32 rotates together with the power input member 31. Also, as the roller base 23 is affixed to the roller case 32 by the screws 27 as mentioned above, the power input member 31, the roller case 32, the roller base 23 and the plucking roller 21 rotate as a single structure on the axis R.

The crown gear 33 of each epilation unit 1 is fitted with the pair of pins 13. These pins 13 are fitted into pin holes 12a formed in the sleeve 12 and into later-described slots 2a formed in the supportive housing 2, so that the crown gear 33 is a nonrotating element. Further, the crown gear 33 has a face gear portion 33a which meshes with the epilation unit-side gear 34. The epilation unit-side gear 34 is pivotably supported by a gear shaft 36 fitted between a side portion of the roller case 32 and a gear cover 38. In the epilation unit 1 thus configured, the epilation unit-side gear 34 turns along the face gear portion 33a of the crown gear 33 when the roller case 32 rotates together with the power input member 31.

The other epilation unit-side gear 35 mates with the aforementioned epilation unit-side gear 34 and is pivotably supported by a gear shaft 37 fitted on a side portion of the roller base 23. This epilation unit-side gear 35 also mates with an unillustrated gear portion formed on an end portion of the plucking roller 21 and thus serves to transmit the rotational driving force to the plucking roller 21 so that the plucking roller 21 can rotate on the roller shaft 22.

The sleeve 12 of each epilation unit 1 surrounds the plucking roller 21 and projects toward the skin surface S. In this embodiment, the sleeve 12 is configured such that an outer end 12b thereof placed against the skin surface S becomes flush with an extreme outer part of the plucking roller 21 that is brought into contact with the skin surface S as shown in FIG. 4. Thus, the individual sleeves 12 go into contact with the skin surface S together with the plucking rollers 21. This arrangement of the embodiment may be modified such that the outer ends 12b of the sleeves 12 slightly project further outward toward the skin surface S than the extreme outer parts of the plucking rollers 21.

The aforementioned supportive housing 2 has a triple-cup structure for accommodating the three epilation units 1. The aforementioned slots 2a in which the pairs of pins 13 of the individual epilation units 1 are fitted are formed in the supportive housing 2 at appropriate points thereof so that each epilation unit 1 can swing in a direction marked by “A” in FIG. 9 with respect to the supportive housing 2. Also, the slots 2a in the supportive housing 2 are formed to create some play for the pins 13 of each epilation unit 1 to move along a direction marked by “B” in FIG. 9 so that each epilation unit 1 can float (i.e., protrude and retract) along the B direction with respect to the supportive housing 2. This arrangement of the embodiment enables the individual epilation units 1 to float up and down as shown in FIG. 10 while swinging independently of one another.

Referring to FIG. 7, the first motive power transmission mechanism 4 includes driving and transmission gears 41-45 and gear shafts 46-49 rotatably supporting the gears 42-45, respectively, which are arranged in this order from a driving side to a driven side. The first motive power transmission mechanism 4 further includes a gear cover 51, the aforementioned cylindrical members 52 and compression coil springs 53.

The driving gear 41 is fitted on a driving shaft 3a of the motor 3. The gears 42-44 are rotatably supported by the respective gear shafts 46-48 of which opposite ends are supported by the supportive housing 2 and the gear cover 51. With the first motive power transmission mechanism 4 thus configured, the motive power of the motor 3 output from the driving shaft 3a is transmitted to the individual epilation units 1 through three driving gears 45. The aforementioned gear cover 51 is fixed to an inside of a recessed part 6a of the outer cover 6 by screws 8.

The three driving gears 45 fitted on the gear shafts 49 which are mounted in the supportive housing 2 are configured to engage the respective cylindrical members 52. Specifically, as gear teeth formed on a curved outer surface of a projecting sleevelike part 45a of each driving gear 45 mesh with gear teeth formed on a curved inner surface of a sleevelike part 52a of the corresponding cylindrical member 52 as shown in FIG. 5, the motive power of the motor 3 is transmitted to each cylindrical member 52. The compression coil spring 53 is fitted in the sleevelike part 52a of the cylindrical member 52 of each epilation unit 1 to produce a biasing force acting on the gear shaft 45 and the cylindrical member 52 in opposite directions, causing both to separate from each other. This configuration makes it possible to easily bring each epilation unit 1 which has retracted into the supportive housing 2 back to an original position.

Each of the cylindrical members 52 of the first motive power transmission mechanism 4 has the aforementioned projecting part 52b which meshes with the recessed mating part 31a of the corresponding power input member 31. The projecting part 52b of each cylindrical member 52 has a generally triangular shape in plan view. The mating part 31a of each power input member 31 also has a generally triangular shape in plan view although not illustrated. This arrangement ensures that the epilation units 1 will not easily be disengaged from the supportive housing 2 regardless of floating and/or swinging action of the epilation units 1 relative to the supportive housing 2 (refer to FIG. 11).

In the hair removal apparatus 100 thus configured, the motive power (rotational driving force) produced by the motor 3 is transmitted to the three driving gears 45 through the gears 41-45 of the first motive power transmission mechanism 4 (refer to FIG. 7) and then further downstream to the power input members 31 of the epilation units 1 via the respective cylindrical members 52 (refer to FIG. 8), thereby causing the rotors 11 of the epilation units 1 to rotate on the respective axes R.

Since the cylindrical members 52 of the first motive power transmission mechanism 4 are connected to the power input members 31 with the projecting parts 52b flexibly meshed with the respective mating parts 31a as mentioned above, the cylindrical members 52 and the power input members 31 are securely held in an interconnected state even when the epilation units 1 are making swing motion in the supportive housing 2. Additionally, since the cylindrical members 52 incorporate the compression coil springs 53 for biasing the cylindrical members 52 toward the respective power input members 31, the cylindrical members 52 can float up and down relative to the respective driving gears 45. This structure of the embodiment makes it possible to reliably transmit the motive power (rotational driving force) to the epilation units 1 even when the epilation units 1 swing and float to a great extent relative to the supportive housing 2.

Since the epilation unit-side gears 34 built in the rotors 11 mesh with the face gear portions 33a of the respective crown gears 33, the epilation unit-side gears 34 rotate on their own axes when the rotors 11 turn on the respective axes R. Also, as the rotational driving force of the first motive power transmission mechanism 4 is transmitted to the individual plucking rollers 21 via the epilation unit-side gears 34, 35, the plucking rollers 21 rotate on the respective roller shafts 22.

As thus far described, the three epilation units 1 incorporate the rotors 11 of which plucking rollers 21 turn on the respective axes R while rotating on the respective roller shafts 22. With this arrangement, the hair removal apparatus 100 of the embodiment can pluck the body hairs from the skin. As the individual epilation units 1 can swing and float independently of one another according to swelling and hollowed features of the skin surface S, the hair removal apparatus 100 can epilate body hairs with the three epilation units 1 held in sufficiently close contact with the skin surface S.

Since the hair removal apparatus 100 of the present embodiment is configured such that the three epilation units 1 float, or protrude and retract, independently of one another as much as heights and depths of swells and hollows of the skin surface S as mentioned above, it is possible to pull out body hairs from the skin surface S having swollen and hollowed parts over sufficiently broad areas of contact between the epilation units 1 and the skin surface S. In this structure, the hair removal apparatus 100 provided with the three epilation units 1 can treat an increased epilation area yet preventing a reduction in epilation efficiency.

In this embodiment, the three epilation units 1 are configured to swing independently of one another as mentioned above. Since the individual epilation units 1 swing in appropriate directions so that the epilation units 1 fit swollen and hollowed areas of the skin surface S, it is possible to keep the epilation units 1 in close contact with the skin surface S having swollen and hollowed parts over sufficiently broad skin areas. This structure sufficiently prevents a reduction in epilation area.

In the hair removal apparatus 100 of the foregoing embodiment, the pins 13 of each epilation unit 1 are fitted in the slots 2a formed in the supportive housing 2 as mentioned above. This simple structure allows the individual epilation units 1 to float and swing independently of one another with respect to the supportive housing 2.

In this embodiment, the cylindrical member 52 of each epilation unit 1 is made floatable up and down relative to the supportive housing 2 and the cylindrical member 52 and the power input member 31 are flexibly connected to each other as mentioned above. This structure allows each epilation unit 1 to float and swing relative to the supportive housing 2 while transmitting the motive power (rotational driving force) from the first motive power transmission mechanism 4 to the second motive power transmission mechanism 25 in a reliable fashion.

Furthermore, the cylindrical member 52 of each epilation unit 1 is biased in the direction toward the skin surface S by the compression coil spring 53 in such a manner that each epilation unit 1 can retract into the supportive housing 2 overwhelming the biasing force of the compression coil spring 53 when the plucking roller 21 is acted upon by a proper level of reaction force from the skin surface S. In this structure, each epilation unit 1 is automatically brought back to the original position by the biasing force of the compression coil spring 53 when the reaction force from the skin surface S is removed.

In this embodiment, the individual sleeves 12 go into contact with the skin surface S together with the plucking rollers 21. This arrangement is advantageous for preventing such inconvenience that the hair removal apparatus 100 is led to unintended directions on the skin surface S due to strong contact of only the rotating plucking rollers 21 with the skin, thereby enabling smooth epilation. Additionally, this arrangement of the embodiment serves to alleviate an unpleasant feeling that the user may have when the hair is pulled out of the skin because the sleeves 12 hold the skin tight during epilation.

While the invention has thus been described with reference to the hair removal apparatus 100 of the preferred embodiment provided with the three epilation units 1, the invention is not limited to this embodiment but is similarly applicable to a hair removal apparatus provided with one or two epilation units or more than three epilation units.

The invention has thus far been described in detail, by way of example, with reference to the preferred embodiment thereof.

In summary, a hair removal apparatus comprises a plurality of extractors each of which rotates on a first rotary axis extending in a direction generally parallel to a skin surface during epilation, each of the extractors having a tweezing mechanism for grasping hairs, and a driver for turning each of the extractors on a second rotary axis which is kept generally perpendicular to the skin surface. The plurality of extractors are configured to protrude and retract in a direction generally perpendicular to the skin surface independently of one another, and the hair removal apparatus pulls the grasped hairs out of the skin surface when moved with the extractors held in contact with the skin surface.

Since the hair removal apparatus is configured such that the plurality of extractors float, or protrude and retract, independently of one another as much as heights and depths of swells and hollows of the skin surface, it is possible to pull out body hairs from the skin surface having swollen and hollowed parts over sufficiently broad areas of contact between the extractors and the skin surface. In this structure, the hair removal apparatus provided with the plurality of extractors can treat an increased epilation area yet preventing a reduction in epilation efficiency.

The hair removal apparatus may be configured such that the plurality of extractors are swingable in directions along the skin surface independently of one another while protruding and retracting in the direction generally perpendicular to the skin surface independently of one another.

In the hair removal apparatus thus configured, the plurality of extractors can swing independently of one another. Since the individual extractors swing in appropriate directions so that the extractors fit swollen and hollowed areas of the skin surface, it is possible to keep the extractors in close contact with the skin surface having swollen and hollowed parts over sufficiently broad skin areas. This structure sufficiently prevents a reduction in epilation area.

The hair removal apparatus may be configured such that the driver includes a first motive power transmission mechanism connected to a power source, and a plurality of second motive power transmission mechanisms connected to the first motive power transmission mechanism to transmit a driving force supplied therefrom to the individual extractors, wherein each of the extractors and the corresponding second motive power transmission mechanism are assembled to constitute a modular epilation unit. The hair removal apparatus may be further provided with a casing for supporting the epilation units, the casing being provided with a pin which is fitted in a slot formed in the casing so that each of the epilation units can protrude and retract, and swing as the pin moves along and turns in the slot.

According to this feature, it is possible to allow the individual epilation units to float and swing independently of one another with respect to the casing with a simple structure using pins and slots.

The hair removal apparatus may be configured such that the first motive power transmission mechanism has an output portion which is made movable relative to the casing in the direction generally perpendicular to the skin surface, and each of the second motive power transmission mechanisms has an input portion which flexibly meshes with the output portion of the first motive power transmission mechanism.

This structure allows each epilation unit to float and swing relative to the casing while transmitting motive power (rotational driving force) from the first motive power transmission mechanism to the second motive power transmission mechanisms in a reliable fashion.

The first motive power transmission mechanism may be provided with a biasing member which biases the output portion of the first motive power transmission mechanism in a direction toward the skin surface.

According to this feature, each epilation unit can retract into the casing overwhelming a biasing force exerted by the biasing member when the extractor is acted upon by a proper level of reaction force from the skin surface. In this structure, each epilation unit is automatically brought back to an original position by the biasing force of the biasing member when the reaction force from the skin surface is removed.

The hair removal apparatus may be further provided with a sleeve which surrounds the extractors and projects outward toward the skin surface at least to a point where an outer end of the sleeve becomes flush with an extreme outer part of each of the extractors.

In this structure, the sleeve goes into contact with the skin surface together with the extractors. This arrangement is advantageous for preventing such inconvenience that the hair removal apparatus is led to unintended directions on the skin surface due to strong contact of only the rotating extractors with the skin, thereby enabling smooth epilation. Additionally, this arrangement serves to alleviate an unpleasant feeling that the user may have when the hair is pulled out of the skin because the sleeves hold the skin tight during epilation.

This application is based on patent application No. 2007-43360 filed in Japan, the contents of which are hereby incorporated by references.

As this invention may be embodied in several forms without departing from the spirit of essential characteristics thereof, the present embodiment is therefore illustrative and not restrictive, since the scope of the invention is defined by the appended claims rather than by the description preceding them, and all changes that fall within metes and bounds of the claims, or equivalence of such metes and bounds are therefore intended to embraced by the claims.

What is claimed is:

1. A hair removal apparatus comprising:
a plurality of extractors each of which rotates on a first rotary axis extending in a direction generally parallel to a skin surface during epilation, each of the extractors having a tweezing mechanism for grasping hairs; and
a driver for turning each of the extractors on a second rotary axis which is kept generally perpendicular to the skin surface;
wherein the plurality of extractors are configured to protrude and retract in a direction generally perpendicular to the skin surface independently of one another, and the hair removal apparatus pulls the grasped hairs out of the skin surface when moved with the extractors held in contact with the skin surface.

2. The hair removal apparatus according to claim 1, further comprising a sleeve which surrounds the extractors and projects outward toward the skin surface at least to a point where an outer end of the sleeve becomes flush with an extreme outer part of each of the extractors.

3. The hair removal apparatus according to claim 1, wherein the driver includes:
a first motive power transmission mechanism connected to a power source; and
a plurality of second motive power transmission mechanisms connected to the first motive power transmission mechanism to transmit a driving force supplied therefrom to the individual extractors;
wherein each of the extractors and the corresponding second motive power transmission mechanism are assembled to constitute a modular epilation unit;
the hair removal apparatus further comprising:
a casing for supporting the epilation units, the casing being provided with a pin which is fitted in a slot formed in the casing so that each of the epilation units can protrude and retract, and swing as the pin moves along and turns in the slot.

4. The hair removal apparatus according to claim 1, wherein the plurality of extractors are swingable in directions along the skin surface independently of one another while protruding and retracting in the direction generally perpendicular to the skin surface independently of one another.

5. The hair removal apparatus according to claim 4, further comprising a sleeve which surrounds the extractors and projects outward toward the skin surface at least to a point where an outer end of the sleeve becomes flush with an extreme outer part of each of the extractors.

6. The hair removal apparatus according to claim 4, wherein the driver includes:
a first motive power transmission mechanism connected to a power source; and
a plurality of second motive power transmission mechanisms connected to the first motive power transmission mechanism to transmit a driving force supplied therefrom to the individual extractors;
wherein each of the extractors and the corresponding second motive power transmission mechanism are assembled to constitute a modular epilation unit;
the hair removal apparatus further comprising:
a casing for supporting the epilation units, the casing being provided with a pin which is fitted in a slot formed in the casing so that each of the epilation units can protrude and retract, and swing as the pin moves along and turns in the slot.

7. The hair removal apparatus according to claim 6, further comprising a sleeve which surrounds the extractors and projects outward toward the skin surface at least to a point where an outer end of the sleeve becomes flush with an extreme outer part of each of the extractors.

8. The hair removal apparatus according to claim 6, wherein the first motive power transmission mechanism has an output portion which is made movable relative to the casing in the direction generally perpendicular to the skin surface, and each of the second motive power transmission mechanisms has an input portion which flexibly meshes with the output portion of the first motive power transmission mechanism.

9. The hair removal apparatus according to claim 8, further comprising a sleeve which surrounds the extractors and projects outward toward the skin surface at least to a point where an outer end of the sleeve becomes flush with an extreme outer part of each of the extractors.

10. The hair removal apparatus according to claim 8, wherein the first motive power transmission mechanism is provided with a biasing member which biases the output portion of the first motive power transmission mechanism in a direction toward the skin surface.

11. The hair removal apparatus according to claim 10, further comprising a sleeve which surrounds the extractors and projects outward toward the skin surface at least to a point where an outer end of the sleeve becomes flush with an extreme outer part of each of the extractors.
